(19) United States
(12) Patent Application Publication
(10) Pub. No.: US 2023/0320987 A1
(43) Pub. Date: Oct. 12, 2023

(54) LIPID NANOPARTICLES AND LIPOSOMES

(71) Applicants: National Health Research Institutes, Miaoli County (TW); National Tsing Hua University, Hsinchu (TW)

(72) Inventors: Yunching Chen, Hsinchu City (TW); Kak-Shan Shia, Taipei City (TW); Chiung-Tong Chen, New Taipei City (TW); Chien-Huang Wu, New Taipei City (TW); Ya-Ping Chen, Taichung City (TW)

(21) Appl. No.: 18/295,918

(22) Filed: Apr. 5, 2023

Related U.S. Application Data
(60) Provisional application No. 63/328,006, filed on Apr. 6, 2022.

(51) Int. Cl. A61K 9/16; A61K 31/421; A61K 47/54; A61K 31/05

(52) U.S. Cl. CPC A61K 9/1617; A61K 31/421; A61K 47/544; A61K 31/05

(57) ABSTRACT

Disclosed is a pharmaceutical nanoparticle containing a core and a shell coating the core. The core contains (3-{4-[2-({4-[3-(3-cyclohexylamino-propylamino)-propyl]-oxazol-2-ylmethyl}-amino)-6-methyl-pyrimidin-4-ylamino]-piperidin-1-yl}-3-oxo-propylamino)-acetic acid or a salt thereof, 1,2-dioleoyl-sn-glycero-3-phosphate, and an anionic polymer. The shell contains a lipid. Also disclosed is a method for preparing such as pharmaceutical nanoparticle. Further provided are a liposome containing a lipid bilayer enclosing an aqueous core and its preparation method.

### CROSS REFERENCE TO RELATED APPLICATION

This application claims priority to U.S. Provisional Application No. 63/328,006 filed on Apr. 6, 2022, the content of which is hereby incorporated by reference in its entirety.

### BACKGROUND

Type 4 CXC chemokine receptor (“CXCR4”) antagonists are useful in treating various disorders such as hepatocellular carcinoma, rheumatoid arthritis, kidney injury, myocardial infarction, and mild traumatic brain injury.

Promising CXCR4 antagonists have been discovered, including (3-{4-[2-({4-[3-(3-cyclohexylaminopropylamino)propyl]oxazol-2-ylmethyl}amino)-6-methyl-pyrimidin-4-ylamino]piperidin-1-yl}-3-oxopropylamino)acetic acid (“CX-1”). See U.S. Pat. No. 10,882,854.

Due to its high water solubility, CX-1 has a half-life as short as less than one hour after subcutaneous injection into a human body. The short half-life presents challenges in commercializing CX-1 as a useful pharmaceutical drug. Given its fast removal from the body, CX-1 therapeutic benefits are difficult to achieve without frequent administration, e.g., more than three times a day.

Delivery systems have been designed to both protect a pharmaceutical drug from quick metabolization and release it slowly to the blood stream, thus addressing issues stemming from a short half-life. To ensure effective protection and controlled release, a specific delivery system must be developed for each drug due to its unique physiochemical properties. Currently, no delivery system has been reported for CX-1.

There is a need to develop a delivery system that protects CX-1 from metabolization and releases it in a controlled manner.

### SUMMARY

Within the scope of the present invention are effective delivery systems for CX-1 that maintain drug levels within a desired therapeutic window for a long period of time. The CX-1 administration frequency is reduced from more than three times a day to as low as three times a week.

In one aspect, this invention relates to a pharmaceutical nanoparticle containing a core and a shell coating the core, in which the core includes (3-{4-[2-({4-[3-(3-cyclohexylaminopropylamino)propyl]oxazol-2-ylmethyl}amino)-6-methyl-pyrimidin-4-ylamino]piperidin-1-yl}-3-oxopropylamino)acetic acid (“CX-1”), 1,2-dioleoyl-sn-glycero-3-phosphate (“DOPA”), and an anionic polymer; and the shell includes one or more lipids.

The nanoparticle can have one or both of the following features: (i) a particle size of 1 nm to 1000 nm (e.g., 10 nm to 500 nm and 100 nm to 300 nm) and (ii) a zeta potential of 0 mV to −100 mV (e.g., −1 mV to −50 mV and −5 mV to −30 mV).

Optionally, the core further contains 1,2-dioleoyl-3-trimethylammonium-propane (“DOTAP”), in addition to CX-1, DOPA, and the anionic polymer.

CX-1 is encapsulated in the pharmaceutical nanoparticle as the compound itself or as a salt. Examples of a CX-1 salt include a hydrochloride salt, a hydrobromide salt, a citric acid salt, a maleic acid salt, a diphosphate salt, and combinations thereof.

The weight ratio between CX-1 and the lipid shell is typically 1:80 to 4:1 (e.g., 1:40 to 2:1 and 1:20 to 1:1).

A preferred anionic polymer is a calf thymus deoxyribonucleic acid (“DNA”), a polyphenol, cyclic guanosine monophosphate-adenosine monophosphate (“cGAMP”), a small interfering ribonucleic acid (“siRNA”), a plasmid DNA, or any combination thereof.

When the anionic polymer is a calf thymus DNA, the core contains CX-1, DOPA, and the calf thymus DNA at a weight ratio of CX-1:DOPA:calf thymus DNA being 1:(0.01-100):(0.01-100), e.g., 1:(0.05-20):(0.05-20) and 1:(1-20):(0.4-1).

A polyphenol is also a suitable anionic polymer. Examples include tannic acid, 1,2,3,4,6-pentagalloyl glucose, epigallocatechin gallate, β-glucogallin, 3,4,5-trihydroxybenzoic acid, theaflavin-3-gallatt, raspberry ellagitannin, acertannin, hamamelitannin, and combinations thereof.

When the core contains tannic acid as the anionic polymer, the weight ratio of CX-1:DOPA:tannic acid is preferably 1:(0.01-100):(0.01-100), preferably, 1:(0.05-20):(0.05-20) and 1:(1-4):(1-10).

Turning to the shell of the pharmaceutical nanoparticle, it contains a lipid, e.g., cholesterol, DOPA, DOTAP, 1,2-dioleoyl-sn-glycero-3-phosphocholine (“DOPC”), 1,2-dioleoyl-sn-glycero-3-phosphoethanolamine (“DOPE”), 1,2-distearoyl-sn-glycero-3-phosphoethanolamine-N-[methoxy(polyethylene glycol)] (“DSPE-PEG”), s poly(D,L-lactide-co-glycolide) (“PLGA”), or any combination thereof. One specific example of DSPE-PEG is 1,2-distearoyl-sn-glycero-3-phosphoethanolamine-N-[methoxy(polyethylene glycol)-2000] (“DSPE-PEG2000”). In some embodiments, the shell contains one or more lipids selected from the group consisting of DSPE-PEG2000, DOPC, DOTAP, cholesterol, and PLGA, with the weight ratio of DSPE-PEG2000:DOPC:DOTAP:cholesterol:PLGA being 4:(0-10):(0-10):(0-10):(0-10), preferably 4:(0.2-5):(0.2-5):(0.2-5):(0-5), and more preferably 4:(0.5-2):(0.5-2):(0.5-2):(0-0.2). In other embodiments, the shell contains one or more lipids selected from the group consisting of DSPE-PEG2000, DOPC, DOPA, cholesterol, and PLGA, with the weight ratio of DSPE-PEG2000:DOPC:DOPA:cholesterol:PLGA being 4:(0-10):(0-10):(0-10):(0-10), preferably 4:(0.2-5):(0.2-5):(0.2-5):(0-5), and more preferably 4:(0.5-2):(0.5-2):(0.5-2):(0-0.2).

Also within the scope of this invention is a method of preparing any of the pharmaceutical nanoparticles described above. The method includes at least the steps of: (1) providing a core dispersion having cores dispersed in a solvent, the cores each containing CX-1, DOPA, and an anionic polymer; (2) providing a lipid; and (3) mixing the core dispersion and the lipid, thereby coating each of the cores with the lipid.

In another aspect, the invention relates to a liposome containing a lipid bilayer enclosing an aqueous core. The lipid bilayer contains 1,2-distearoyl-sn-glycero-3-phosphocholine (“DSPC”), cholesterol, and DSPE-PEG2000. The aqueous core contains CX-1 or a salt thereof.

The liposome typically has a particle size in diameter of 30 nm to 300 nm (e.g., 100 nm to 200 nm and 140 nm to 160 nm) and a zeta potential of 0 mV to −20 mV (e.g., −1 mV to −15 mV and −2 mV to −10 mV).

The weight ratio of CX-1:DSPC:cholesterol:DSPE-PEG2000 is preferably in the range of 1:(0.5-12):(0.1-4):(0.02-1), e.g., 1:(4-10):(0.5-2.5):(0.1-0.5) and 1:(6-8):(1.5-2):(0.3-0.4).

Still within the scope of this invention is a method of preparing any of the above-described liposomes. The method includes the steps of: (i) providing a thin film containing DSPC, cholesterol, and DSPE-PEG2000, (ii) mixing the thin film with an aqueous solution containing CX-1 (e.g., at a concentration of 0.3% to 8% by weight of the aqueous solution) and ammonium sulfate (e.g., at a concentration of 1% to 6% by weight of the aqueous solution) to obtain a hydration mixture, and (iii) freezing the hydration mixture to a temperature of −150° C. to −200° C. and then thawing it to a temperature of 50° C. to 75° C. to obtain a dispersion containing the liposome. The freezing-thawing step can be repeated 4 to 10 times.

Further, the method can include the additional step of extruding the dispersion through a membrane having a pore diameter of 30 nm to 400 nm. The membrane is preferably formed of polycarbonate.

The term “CX-1” herein includes CX-1, its salts, solvates, and prodrugs. A salt can be formed between an anion and a positively charged group (e.g., amino) on CX-1. Examples of a suitable anion are chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. A salt can also be formed between a cation and a negatively charged group. Suitable cations include sodium ion, potassium ion, magnesium ion, calcium ion, and ammonium cation such as tetramethyl-ammonium ion. Further, a salt can contain quaternary nitrogen atoms. A solvate refers to a complex formed between CX-1 and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active CX-1. Examples of a prodrug include esters and other pharmaceutically acceptable derivatives.

The term “treating” or “treatment” refers to administering one or more of the compounds to a subject, who suffers from a disorder including hepatocellular carcinoma, rheumatoid arthritis, kidney injury, myocardial infarction, or mild traumatic brain injury, or has a predisposition toward one of them, with the purpose to confer a therapeutic effect, e.g., to cure, relieve, alter, affect, ameliorate, or prevent such a disorder, symptoms, or the predisposition.

“An effective amount” refers to the amount of CX-1 in a composition that is required to confer therapeutic effect. Effective doses will vary, as recognized by those skilled in the art, depending on type of symptoms treated, route of administration, excipient usage, and the possibilities of co-usage with another therapeutic treatment.

To practice the method of the present invention, a pharmaceutical composition containing one or more of the above-described nanoparticles or liposomes can be administered parenterally, orally, nasally, rectally, topically, or buccally.

The term “parenteral” as used herein refers to subcutaneous, intracutaneous, intravenous, intraperitoneal, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or di-glycerides). Fatty acid, such as oleic acid and its glyceride derivatives, are useful in the preparation of injectables, as are natural pharmaceutically acceptable oils, such as olive oil and castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens and Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents.

A composition can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound. Examples include colloidal silicon oxide, magnesium stearate, cellulose, sodium lauryl sulfate, and D&C Yellow #10.

The details of one or more embodiments of the invention are set forth in the description below. Other features, objects, and advantages of the invention will be apparent from the description and from the claims.

### DETAILED DESCRIPTION

To address issues related to a very short half-life of CX-1, delivery systems are provided for encapsulating CX-1 in a pharmaceutical nanoparticle or a liposome.

Such a delivery system releases CX-1 gradually following administration, thus providing long-acting therapeutic benefits and avoiding hazardous peak concentrations.

### CXCR4 Antagonist CX-1

As shown below, CX-1 contains five secondary amino groups (—NH—), a pyrimidine ring, an oxazole ring, and a carboxylic acid group (—COOH).

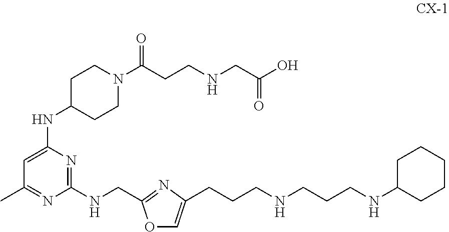 [SMILES: Cc1cc(NC2CCN(C(=O)CCNCC(=O)O)CC2)nc(NCc2nc(CCCNCCCNC3CCCCC3)co2)n1]

As such, it can be negatively charged through the carboxylic acid group or positively charged through the amino groups, the pyrimidine ring, or the oxazole ring. CX-1 is encapsulated in the delivery system as the compound itself or as a salt. Diphosphate is a preferred salt, which is readily obtained by mixing two moles of phosphate acid with each mole of CX-1. Other suitable CX-1 salts include hydrochloride salt, hydrobromide salt, citric acid salt, and maleic acid salt. They can be readily prepared from CX-1 and a corresponding salt following conventional methods.

The preparation of CX-1 is described in detail in U.S. Pat. No. 10,882,854. Other well-known synthetic methods in the art can also be applied to obtain CX-1. See, e.g., R. Larock, Comprehensive Organic Transformations (3^{rd} Ed., John Wiley and Sons 2018); P. G. M. Wuts and T. W. Greene, Greene's Protective Groups in Organic Synthesis (4^{th} Ed., John Wiley and Sons 2007); L. Fieser and M. Fieser, Fieser and Fieser's Reagents for Organic Synthesis (John Wiley and Sons 1994); and L. Paquette, ed., Encyclopedia of Reagents for Organic Synthesis (2^{nd} ed., John Wiley and Sons 2009) and subsequent editions thereof.

### Pharmaceutical Nanoparticles

The pharmaceutical nanoparticles of this invention each encapsulate CX-1 in a core covered by a lipid shell. In addition to CX-1, the core also contains an anionic lipid (i.e., DOPA) and an anionic polymer so that CX-1 is effectively entrapped and protected. In some embodiments, DOTAP, a cationic lipid, is optionally added to the core in addition to or as a replacement of DOPA.

A lipid shell coating the core provides additional entrapment and protection using one or more lipids that is compatible with CX-1. More importantly, the lipid shell stabilizes the nanoparticle.

In some pharmaceutical nanoparticles of this invention, the core contains by weight (i) 1% to 80% (e.g., 2% to 70%, 3% to 60%, and 4% to 55%) of CX-1, (ii) 1% to 60% (e.g., 2% to 50%, 3% to 40%, and 4% to 30%) of an anionic polymer (e.g., calf thymus DNA), and (iii) 10% to 98% (e.g., 20% to 96%, 30% to 95%, and 40% to 92%) of DOPA, DOTAP, or their combination.

In other pharmaceutical nanoparticles of this invention, the weight ratio of CX-1:DOPA/DOTAP:calf thymus DNA is 1:(0.01-100):(0.01-100), preferably 1:(0.05-20):(0.05-20), and more preferably 1:(1-20):(0.4-1).

In still other pharmaceutical nanoparticles, the core contains by weight (i) 1% to 50% (e.g., 3% to 40%, 6% to 30%, and 8% to 20%) of CX-1, (ii) 10% to 95% (e.g., 20% to 90%, 30% to 80%, and 40% to 75%) of an anionic polymer (e.g., tannic acid), and (iii) 2% to 60% (e.g., 5% to 50%, 10% to 50%, and 15% to 40%) of DOPA, DOTAP, or their combination. Alternatively, the weight ratio of CX-1:DOPA/DOTAP:tannic acid is 1:(0.01-100):(0.01-100), preferably 1:(0.05-20):(0.05-20), and more preferably 1:(1-4):(1-10).

DOPA is commercially available as a sodium salt from various suppliers, e.g., Millipore Sigma (Burlington, Massachusetts).

DOTAP can be purchased as a chloride salt from Avanti Polar Lipids, Birmingham, Alabama.

Suppliers for a calf thymus DNA solution include Sigma-Aldrich (St. Louis, Missouri) and ThermoFisher Scientific (Waltham, Massachusetts).

Tannic acid is a polyphenol extracted from certain woody flowering plants and food such as fruits, nuts, wine, and tea. Its chemical formula is C₇₆H₅₂O₄₆, corresponding to decagalloyl glucose. A representative structure is shown below.

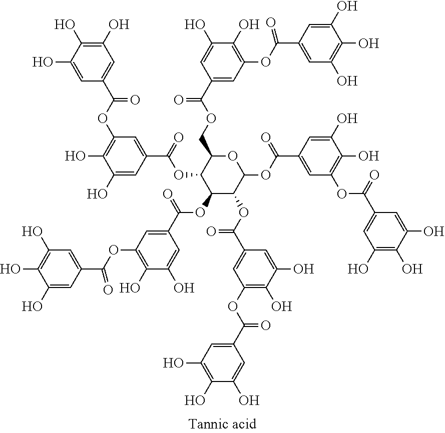 [SMILES: O=C(OC[C@H]1OC(OC(=O)c2cc(O)c(O)c(OC(=O)c3cc(O)c(O)c(O)c3)c2)[C@H](OC(=O)c2cc(O)c(O)c(OC(=O)c3cc(O)c(O)c(O)c3)c2)[C@@H](OC(=O)c2cc(O)c(O)c(OC(=O)c3cc(O)c(O)c(O)c3)c2)[C@@H]1OC(=O)c1cc(O)c(O)c(OC(=O)c2cc(O)c(O)c(O)c2)c1)c1cc(O)c(O)c(OC(=O)c2cc(O)c(O)c(O)c2)c1]

Tannic acid is a mixture of polygalloyl glucoses or polygalloyl quinic acid esters with the number of galloyl moieties per molecule ranging from 2 to 12 depending on the plant source used to extract the tannic acid. Suppliers include Sigma-Aldrich and ThermoFisher Scientific.

In addition to tannic acid and the calf thymus DNA, other suitable anionic polymers include various polyphenols, cyclic guanosine monophosphate-adenosine monophosphate (commercially available from InvivoGen, San Diego, CA, USA), nucleoid acids such as small interfering ribonucleic acids (“siRNA”), and plasmid DNAs. Both siRNAs and plasmid DNAs can be procured from suppliers, e.g., ThermoFisher Scientific and Millipore Sigma.

Polyphenols contain numerous phenol units. They include naturally occurring compounds abundant in plants. Polyphenols include flavonoids (e.g., flavones, flavonols, flavanones, flavanols, isoflavones, catechins, cyanidin, anthocyanins, proanthocyanidins, daidzein, hesperetin, kaempferol, and quercetin), phenolic acids (e.g., polyphenols containing gallic acid moieties, polyphenols containing cinnamic acid moieties, polyphenols containing ferulic acid moieties, and polyphenols containing caffeic acid moieties), polyphenolic amides (e.g., capsaicinoids and avenanthramides), and other polyphenols (e.g., stilbenes, lignans, justicidin A, pinoresinol, matairesinol, secoisolariciresinol, steganacin, podophyllotoxin, resveratrol, pterostilbene, ellagic acid, and curcumin).

Particular suitable polyphenols include those susceptible to negative charges. Examples are provided in Table 1 below with their names, molecular weight information, and structures.

TABLE 1

| | |
|---|---|
| 1,2,3,4,6-pentagalloyl glucose, MW: 941 | Epigallocatechin gallate, MW: 458 |
| 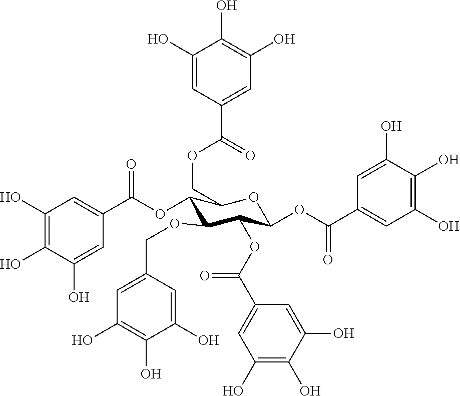 | 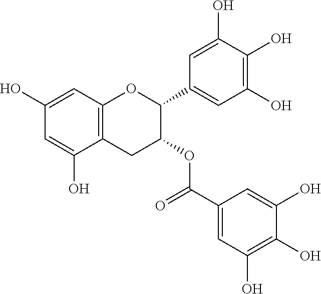 [SMILES: O=C(O[C@@H]1Cc2c(O)cc(O)cc2O[C@@H]1c1cc(O)c(O)c(O)c1)c1cc(O)c(O)c(O)c1] |
| β-Glucogallin, MW: 332 | 3,4,5-trihydroxybenzoic acid, MW: 170 |
| 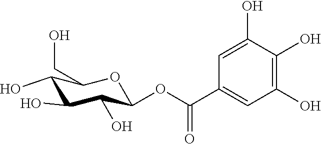 | 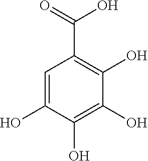 [SMILES: O=C(O)c1cc(O)c(O)c(O)c1O] |
| Theaflavin-3-gallate, MW:717 | Acertannin, MW 468 |
| 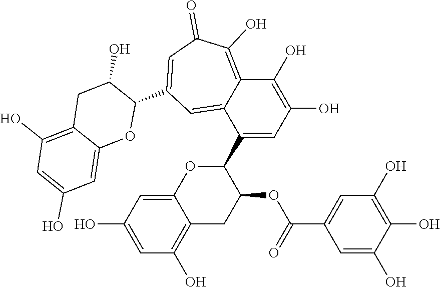 [SMILES: O=C(O[C@H]1Cc2c(O)cc(O)cc2O[C@H]1c1cc(O)c(O)c2c(O)c(=O)cc([C@@H]3Oc4cc(O)cc(O)c4C[C@@H]3O)cc12)c1cc(O)c(O)c(O)c1] | 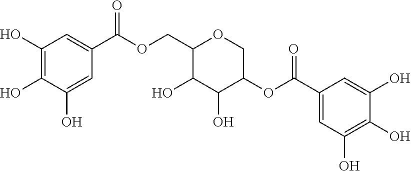 [SMILES: O=C(OCC1OCC(OC(=O)c2cc(O)c(O)c(O)c2)C(O)C1O)c1cc(O)c(O)c(O)c1] |

Hamamelitannin, MW: 484

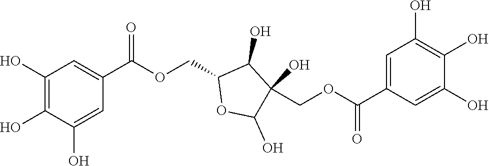 [SMILES: O=C(OC[C@H]1OC(O)[C@@](O)(COC(=O)c2cc(O)c(O)c(O)c2)[C@@H]1O)c1cc(O)c(O)c(O)c1]

Raspberry ellagitannin, MW: 2654

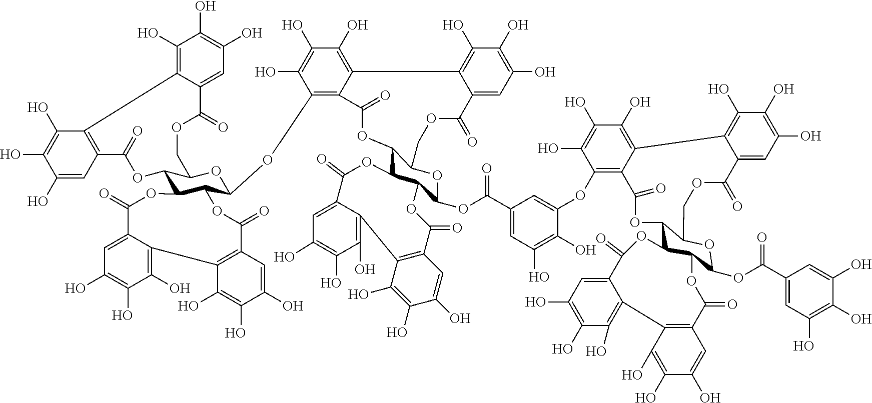

Not wishing to be bound by any theory, it is believed that CX-1, when positively charged through its amino groups or N-containing aromatic rings, bonds to an anionic polymer (e.g., a calf thymus DNA or tannic acid) to form a polymeric complex, which is then covered by a layer of DOPA (i.e., an anionic lipid) to form a core having a particle size of 0.5 nm to 800 nm (e.g., 8 nm to 480 nm and 80 nm to 240 nm).

The term “particle size” refers to the diameter of a core, a nanoparticle, or a liposome. Particle size can be determined by conventional methods such as sieve analysis, dynamic light scattering, high-definition image processing, and passage through an electrically charged orifice.

To further protect CX-1 and stabilize the core, a lipid shell is applied to cover the core to a thickness of 0.1 nm to 950 nm (e.g., 1 nm to 480 nm and 10 nm to 100 nm).

Such a coated nanoparticle of this invention has a particle size of from 1 nm to 1000 nm. In a pharmaceutical composition containing a plurality of nanoparticles, its polydispersity index can be in the range of 0.2 to 0.4.

CX-1 is released from the nanoparticle at a speed determined by multiple factors, the particle size of the core, the particle size of the nanoparticle, the core components and their concentrations, the shell components and their concentrations, the shell thickness, and the weight ratio of CX-1 to the lipid shell. Among them, the weight ratio of CX-1 to the lipid shell plays a major role.

In any nanoparticles described above, the weight ratio of CX-1:the lipid shell typically falls within the range of 1:80 to 4:1.

The lipid shell is formed of either a single lipid or a combination of two or more lipids.

Suitable lipids include cholesterol, DOPA, DOPC, DOPE, DOTAP, PLGA, and DSPE-PEG. The lipid can be anionic, cationic, or non-ionic.

DSPE-PEG is a class of compounds having a 1,2-distearoyl-sn-glycero-3-phosphate moiety connecting to polyethylene glycol (“PEG”) chain through an oxyethylcarbamate group (i.e., —OC₂H₄NHCOO—). Its structure is shown below.

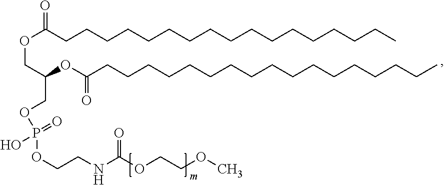

in which m is an integer from 5 to 200 (e.g., 7 to 180, 10 to 150, and 20 to 120).

The molecular weight of a DSPE-PEG depends on the length of the PEG chain. DSPE-PEG useful in this invention has a molecular weight of 500 Daltons to 10,000 Daltons (e.g., 600 Daltons to 8,000 Daltons and 800 Daltons to 6,000 Daltons). DSPE-PEG is commercially available as an ammonium salt. Examples are those from Avanti Polar Lipids, including 1,2-distearoyl-sn-glycero-3-phospho-ethanolamine-N-[methoxy(polyethylene glycol)-350], 1,2-distearoyl-sn-glycero-3-phosphoethanolamine-N-[methoxy(polyethylene glycol)-550], 1,2-distearoyl-sn-glycero-3-phosphoethanolamine-N-[methoxy(polyethylene glycol)-750], 1,2-distearoyl-sn-glycero-3-phosphoethanolamine-N-[methoxy(polyethylene glycol)-1000], 1,2-distearoyl-sn-glycero-3-phosphoethanolamine-N-[methoxy(polyethylene glycol)-2000], 1,2-distearoyl-sn-glycero-3-phosphoethanolamine-N-[methoxy(polyethylene glycol)-3000], and 1,2-distearoyl-sn-glycero-3-phosphoethanolamine-N-[methoxy(polyethylene glycol)-5000].

(Polyethylene glycol) maleimide and poly(ethylene glycol) methyl ether maleimide are also useful lipids to be included in the shell. Their structures are shown below, in which n is an integer from 5 to 200 (e.g., 7 to 180, 10 to 150, and 20 to 120).

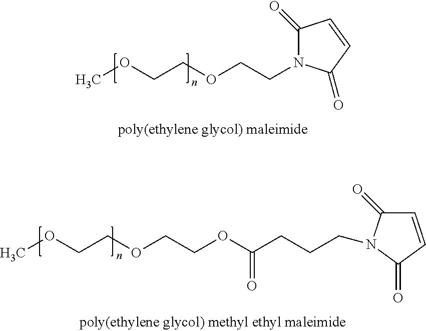

Preferably, the lipid shell is formed of a combination of lipids. Such a combination typically contains DSPE-PEG and one or more additional lipids selected from the group consisting of cholesterol, DOPA, DOPC, DOPE, DOTAP, and PLGA.

Examples of a lipid combination for the shell include:
1. DSPE-PEG2000 and DOTAP
2. DSPE-PEG2000, DOTAP, and cholesterol
3. DSPE-PEG2000, DOTAP, and DOPC
4. DSPE-PEG2000, DOTAP, DOPC, and cholesterol
5. DSPE-PEG2000, DOTAP, and DOPE
6. DSPE-PEG2000, DOTAP, DOPE, and cholesterol
7. DSPE-PEG2000 and DOPA,
8. DSPE-PEG2000, DOPA, and cholesterol
9. DSPE-PEG2000, DOPA, and DOPC
10. DSPE-PEG2000, DOPA, DOPC, and cholesterol
11. DSPE-PEG2000, DOPA, and DOPE
12. DSPE-PEG2000, DOPA, DOPE, and cholesterol
13. DSPE-PEG2000, DOTAP, DOPC, PLGA, and cholesterol
14. DSPE-PEG2000, DOPA, DOPC, PLGA, and cholesterol.

The pharmaceutical nanoparticles of this invention can be prepared following technologies known in the art. See, e.g., Liu et al., The American Society of Gene & Cell Therapy 23, 1772-82 (2015); and Gao et al., Biomaterials 67, 194-203 (2015).

By way of illustration, a pharmaceutical nanoparticle is prepared following the procedure as described below. First, a CX-1 core is prepared from two water-in-oil emulsions. To obtain a first emulsion, an anionic polymer (e.g., tannic acid) aqueous solution is dispersed as aqueous droplets in an oil continuous phase containing DOPA and a first organic solvent. In a second emulsion, a CX-1 aqueous phase is dispersed as aqueous droplets in a second organic solvent. The first and second organic solvents are preferably the same, e.g., a mixture of cyclohexane and polyoxyethylene (5) octylphenyl ether at a volume ratio of 20:1 to 1:10 (e.g., 10:1 to 1:5, 5:1 to 1:1, and 7:3).

The first and second emulsions are then mixed under agitation to form a third emulsion at a temperature of 5° C. to 50° C. (e.g., 10° C. to 40° C. and 15° C. to 35° C.) for 5 minutes to 24 hours (e.g., 10 minutes to 12 hours and 30 minutes to 3 hours). A polymeric complex is first formed in the aqueous phase from ionic bonding between a positively charged CX-1 and the anionic polymer. DOPA is then coated on the surface of the polymeric complex due to its negative charges (forming ionic bonding to positively charged CX-1) and the hydrophilic head (i.e., the phosphate moiety), thereby forming core particles that is a solid or semi-solid homogeneously suspended in the oil phase. The term “semi-solid” refers to an amorphous solid capable of supporting its own weight and holding its shape, while having the ability to flow under pressure.

Ethanol is subsequently added to the third emulsion mixture to precipitate the core particles thus prepared.

Precipitated core particles are isolated from the mixture by a conventional method, e.g., centrifugation. Collection and optionally washing afford core particles with a predetermined particle size as described above. The particle size can be adjusted by varying agitation speed, reaction temperature, concentration of DOPA/CX-1/anionic polymer, organic solvent, ratio of the first emulsion to the second emulsion, etc.

The core particles thus obtained each are subjected to encapsulation by a lipid shell. Before encapsulation, they are first dispersed in a third organic solvent, e.g., chloroform. A lipid is dissolved in a fourth organic solvent (e.g., chloroform) to obtain a lipid solution. An exemplary lipid is a mixture of DOPC, DOPA, DSPE-PEG2000, and cholesterol at a molar ratio of 1:1:1:2. The third organic solvent is miscible with the fourth organic solvent. The third and fourth solvent can be the same or different. Preferably, they are the same.

The core dispersion is subsequently mixed with the lipid solution. Removal of the third and fourth organic solvents yields a plurality of pharmaceutical nanoparticles of this invention.

The pharmaceutical nanoparticles thus prepared can be purified by a conventional method including washing with water or organic solvent, filtration, and extraction.

For convenience of administration, the pharmaceutical nanoparticles are formulated into a pharmaceutical composition, e.g., dispersed in water or any other liquid carriers.

### Liposomes

The liposomes of this invention each can include a lipid bilayer enclosing an aqueous core to form a spherical vesicle, in which the concentric lipid bilayer is formed of DSPC, cholesterol, and DSPE-PEG (e.g., DSPE-PEG2000), and the aqueous core contains CX-1 or its salt.

The weight ratio of CX-1:DSPC:cholesterol:DSPE-PEG is preferably in the range of 1:(0.5-12):(0.1-4):(0.02-1). Certain liposomes of this invention contain by weight 0.1% to 8% (e.g., 0.2% to 6% and 0.5% to 4%) of CX-1, 0.05% to 95% (e.g., 0.1% to 70% and 0.3% to 50%) of DSPC, 0.01% to 30% (e.g., 0.02% to 25% and 0.05% to 15%) of cholesterol, and 0.002% to 8% (e.g., 0.005% to 6% and 0.01% to 4%) of DSPE-PEG.

In the aqueous core, CX-1 is typically present at a level of 0.3% to 8% (e.g., 0.6% to 6% and 1% to 4%) by weight of the core.

Suitable liposomes include multilamellar vesicles each having several lamellar phase lipid bilayers, small unilamellar liposome vesicles each having only one lipid bilayer, large unilamellar vesicles, and cochleate vesicles.

The liposomes of this invention can be prepared following known procedures. See Farzaneh et al., International Journal of Pharmaceutics 551, 300-308 (2018); and Grobmyer et al. (eds.), Cancer Nanotechnology, Methods in Molecular Biology 624 (Springer Science+Business Media, 2010)

An exemplary procedure is provided as follows. A solution of DSPC, cholesterol, and DSPE-PEG2000 in chloroform is dried to form a thin film, which is subsequently hydrated with an ammonium sulfate aqueous solution containing CX-1. The resultant mixture is vortexed at 25° C. for 5 minutes and then shaken at 65° C. for 60 minutes, and then subjected to 2-20 (e.g., 3 to 10, 4 to 8, and 5) freeze-thaw cycles using liquid nitrogen and 65° C. water bath alternatingly, thereby obtaining a sample containing liposomes. To obtain liposomes of uniform size, the sample is extruded through a polycarbonate membrane (pore diameter: 100 nm) for as many as 10-20 times at 65° C., followed by centrifugation through a centrifugal filter (e.g., commercially available under the trade name of 100K Amicon® Ultra 0.5 mL from Millipore, Burlington, Massachusetts) at 4° C. to obtain the final product.

The liposomes thus prepared can be purified by a conventional method including washing with water or organic solvent, filtration, and extraction.

For convenience of administration, the liposomes are formulated into a pharmaceutical composition, e.g., dispersed in a HEPES buffer saline (“HBS”) at a pH value of 7.4 containing 10 mM HEPES and 140 mM NaCl.

Without further elaboration, it is believed that one skilled in the art can, based on the above description, utilize the present invention to its fullest extent. The following examples are to be construed as merely illustrative and not limitative of the remainder of the disclosure in any way whatsoever. All publications cited herein are hereby incorporated by reference in their entirety.

Unless otherwise mentioned, all chemicals are commercially available from Sigma-Aldrich (St. Louis, Missouri).

### Examples A1-A8

### Preparation of CX-1 Nanoparticles Containing a Calf Thymus DNA

Nanoparticle A7 of this invention was prepared following the procedure described below. As a first step, a first emulsion was prepared by mixing at 25° C. for 10 minutes two core materials:(i) 74 μL of a DOPA solution (27 mg/mL in chloroform; Avanti Polar Lipids, Alabaster, Alabama) and (ii) 500 μL of a calf thymus DNA aqueous solution (2 mg/mL; Avanti Polar Lipids) in a solvent containing cyclohexane (4.2 mL, Sigma-Aldrich, St. Louis, Missouri) and branched polyoxyethylene (5) nonylphenylether (1.8 mL; commercially available under the trademark of IGELPAL® CO-520 from Sigma-Aldrich). Subsequently, an aqueous solution (100 μL) of CX-1 diphosphate (10 mg/mL) was added under agitation to the first emulsion at 25° C. The resultant mixture was stirred for 40 minutes to obtain a second emulsion with cores. Ethanol (6 mL) was then added to the second emulsion to precipitate the cores containing CX-1, DOPA, and the calf thymus DNA. After centrifuging at 10000 g for 20 minutes, the cores were collected and then dispersed in chloroform (0.4 mL), which was added to a mixture (2 mg) of DOPC, DOTAP, DSPE-PEG2000, and cholesterol at a molar ration of 1:1:1:2. The resultant mixture was dried under nitrogen gas to obtain a pharmaceutical nanoparticle of this invention, e.g., Nanoparticle A7. Water (500 l) was added to afford a Nanoparticle A7 aqueous dispersion ready for injection.

Seven more pharmaceutical nanoparticles of this invention, i.e., A1-A6 and A8, were prepared following the procedure, supra, except that different amounts of components were added such as CX-1, DOPA, the calf thymus DNA, the solvent (i.e., a mixture of cyclohexane and branched polyoxyethylene (5) nonylphenylether at a volumetric ratio of 7:3), and the lipid (DOPC/DOTAP/DSPE-PEG2000/cholesterol at a molar ratio of 1:1:1:2). See Tables 2 and 2a below.

Table 2 shows the amount of each component. In this table, CX-1 was added as an aqueous solution at a level of 10 mg/mL, the calf thymus DNA was added as an aqueous solution at a concentration of 2 mg/mL, and DOPA was added as an organic solution at a concentration of 27 mg/mL in chloroform. Table 2a shows the weight ratios among the components.

TABLE 2
Nanoparticles A1-A8

| | CX-1 | Calf thymus | DOPA | Solvent | Lipid |
|---|---|---|---|---|---|
| Example | (μL) | DNA (μL) | (μL) | (mL) | (mg) |
| A1 | 50 | 100 | 37 | 0.5 | 1 |
| A2 | 10 | 40 | 74 | 1 | 2 |
| A3 | 50 | 100 | 74 | 6 | 2 |
| A4 | 100 | 200 | 74 | 6 | 2 |
| A5 | 100 | 300 | 74 | 6 | 2 |
| A6 | 100 | 400 | 74 | 6 | 2 |
| A7 | 100 | 500 | 74 | 6 | 2 |
| A8 | 200 | 400 | 74 | 6 | 2 |

TABLE 2a
Weight ratios

Weight ratio

| | | | | | Drug to |
|---|---|---|---|---|---|
| Example | CX-1 | DNA | DOPA | Lipids | lipid ratio |
| A1 | 0.5 | 0.2 | 1 | 1 | 0.5 |
| A2 | 0.05 | 0.04 | 1 | 1 | 0.05 |
| A3 | 0.25 | 0.1 | 1 | 1 | 0.25 |
| A4 | 0.5 | 0.2 | 1 | 1 | 0.5 |
| A5 | 0.5 | 0.3 | 1 | 1 | 0.5 |
| A6 | 0.5 | 0.4 | 1 | 1 | 0.5 |
| A7 | 0.5 | 0.5 | 1 | 1 | 0.5 |
| A8 | 1 | 0.4 | 1 | 1 | 1.0 |

### Examples A9-A15

### Preparation of CX-1 Nanoparticles Containing Tannic Acid

Nanoparticle A14 of this invention was prepared following the procedure as follows. A first emulsion was prepared by mixing at 25° C. for 10 minutes 74 μL of a DOPA solution (27 mg/mL in chloroform) and 40 μL of a tannic acid aqueous solution (120 mg/mL in water) in an organic solvent (3 mL) containing cyclohexane and branched polyoxyethylene (5) nonylphenylether at a volumetric ratio of 7:3. A second emulsion was obtained by emulsifying at 25° C. for 10 minutes 50 μl of a CX-1 aqueous solution (20 mg/mL) in 3 mL of an oil phase containing cyclohexane and branched polyoxyethylene (5) nonylphenylether (7:3, v/v). A mixture of the first and second emulsions was stirred at 25° C. for 10 minutes to obtain a third emulsion. Ethanol (6 mL) was added to the third emulsion to precipitate cores containing CX-1, DOPA, and tannic acid. After centrifuging at 10000 g for 20 minutes, the cores were collected and then dispersed in chloroform. The chloroform dispersion was added to a lipid (2 mg) containing DOPC, DOPA, DSPE-PEG2000, and cholesterol at a molar ratio of 1:1:1:2. Drying under N₂ yielded a pharmaceutical nanoparticle of this invention, e.g., Nanoparticle A14, which was dispersed in 500 μL of water to afford a Nanoparticle A14 aqueous dispersion ready for injection.

Six more pharmaceutical nanoparticles of this invention, i.e., A10-A13 and A15, were prepared following the procedure above except that different amounts of components were used such as CX-1, DOPA, tannic acid, the solvent (cyclohexane and branched polyoxyethylene (5) nonylphenylether at a volumetric ratio of 7:3), and the lipid. See Tables 3 and 3a below. The lipid of Nanoparticles A9-A12 contains DOPC, DOTAP, DSPE-PEG2000, and cholesterol at a molar ratio of 1:1:1:2. The lipid of Nanoparticles A13-A15 contain DOPC, DOPA, DSPE-PEG2000, and cholesterol at a molar ratio of 1:1:1:2.

### Examples A16

### Preparation of CX-1 Nanoparticles Containing Tannic Acid

Nanoparticle A16 of this invention was prepared following the procedure as follows. A first emulsion was prepared by mixing at 25° C. for 10 minutes 74 μL of a DOPA solution (27 mg/mL in chloroform) and 40 μL of a tannic acid aqueous solution (120 mg/mL in water) in an organic solvent (3 mL) containing cyclohexane and branched polyoxyethylene (5) nonylphenylether at a volumetric ratio of 7:3. A second emulsion was obtained by emulsifying at 25° C. for 10 minutes 50 μl of a CX-1 aqueous solution (20 mg/mL) in 3 mL of an oil phase containing cyclohexane and branched polyoxyethylene (5) nonylphenylether (7:3, v/v). A mixture of the first and second emulsions was stirred at 25° C. for 10 minutes to obtain a third emulsion. Ethanol (6 mL) was added to the third emulsion to precipitate cores containing CX-1, DOPA, and tannic acid. After centrifuging at 10000 g for 20 minutes, the cores were collected and then dispersed in chloroform. The chloroform dispersion was added 2 mg a mixture of free lipids (DOPC:DOPA:DSPE-PEG2000:cholesterol=1:1:1:2 molar ratio) and 10 μL of PLGA (75 mg/ml). Drying under N₂ yielded a pharmaceutical nanoparticle of this invention, i.e., Nanoparticle A16, which was dispersed in 500 μL of water to afford a Nanoparticle A16 aqueous dispersion ready for injection.

### Examples A17

### Preparation of CX-1 Nanoparticles Containing Tannic Acid

Nanoparticle A17 of this invention was prepared following the procedure as follows. A first emulsion was prepared by mixing at 25° C. for 10 minutes 74 μL of a DOPA solution (27 mg/mL in chloroform) and 40 μL of a tannic acid aqueous solution (120 mg/mL in water) in an organic solvent (6 mL) containing cyclohexane and branched polyoxyethylene (5) nonylphenylether at a volumetric ratio of 7:3. Subsequently, 50 μl of a CX-1 aqueous solution (20 mg/mL) was added under agitation to the first emulsion at 25° C. The resultant mixture was stirred for 40 minutes to obtain a second emulsion with cores. Ethanol (6 mL) was then added to the second emulsion to precipitate the cores containing CX-1, DOPA, and tannic acid. After centrifuging at 10000 g for 20 minutes, the cores were collected and then dispersed in chloroform. The chloroform dispersion was added 2 mg a mixture of free lipids (DOPC:DOPA:DSPE-PEG2000:cholesterol=1:1:1:2 molar ratio) and 10 μL of PLGA (75 mg/ml). Drying under N₂ yielded a pharmaceutical nanoparticle of this invention, i.e., Nanoparticle A17, which was dispersed in 500 μL of water to afford a Nanoparticle A17 aqueous dispersion ready for injection.

Table 3 shows the amount of each component for Examples A9 to A17. CX-1 was added as an aqueous solution at a level of 20 mg/mL, tannic acid was added as an aqueous solution at a concentration of 120 mg/mL, and DOPA was added as a solution at a concentration of 27 mg/mL in chloroform. The solvent represents the oil phase having cores dispersed therein. Table 3a shows the weigh ratios among the components.

TABLE 3
Pharmaceutical nanoparticles A9-A16

| | CX-1 | Tannic | DOPA | Solvent | Lipid |
|---|---|---|---|---|---|
| Example | (μL) | acid (μL) | (μL) | (mL) | (mg) |
| A9 | 50 | 20 | 74 | 6 | 2^{a} |
| A10 | 50 | 40 | 74 | 6 | 2^{a} |
| A11 | 50 | 50 | 74 | 6 | 2^{a} |
| A12 | 50 | 80 | 74 | 6 | 2^{a} |
| A13 | 50 | 20 | 74 | 6 | 2^{b} |
| A14 | 50 | 40 | 74 | 6 | 2^{b} |
| A15 | 50 | 80 | 74 | 6 | 2^{b} |
| A16 | 50 | 40 | 74 | 6 | 2.75^{c} |
| A17 | 50 | 40 | 74 | 6 | 2.75^{c} |

^{a}The lipid in the shell contains DOPC, DOTAP, DSPE-PEG2000, and cholesterol.
^{b}The lipid in the shell contains DOPC, DOPA, DSPE-PEG2000, and cholesterol.
^{c}The lipid in the shell contains DOPC, DOPA, DSPE-PEG2000, cholesterol, and PLGA.

TABLE 3a
Weight ratios

Weight ratio

| | | Tannic | | | Drug to |
|---|---|---|---|---|---|
| Example | CX-1 | acid | DOPA | Lipid | lipid ratio |
| A9 | 1 | 2.4 | 2 | 2^{a} | 0.5 |
| A10 | 1 | 4.8 | 2 | 2^{a} | 0.5 |
| A11 | 1 | 6 | 2 | 2^{a} | 0.5 |
| A12 | 1 | 7.6 | 2 | 2^{a} | 0.5 |
| A13 | 1 | 2.4 | 2 | 2^{b} | 0.5 |
| A14 | 1 | 4.8 | 2 | 2^{b} | 0.5 |
| A15 | 1 | 7.6 | 2 | 2^{b} | 0.5 |
| A16 | 1 | 4.8 | 2 | 2.75^{c} | 0.36 |
| A17 | 1 | 4.8 | 2 | 2.75^{c} | 0.36 |

^{a}The lipid in the shell contains DOPC, DOTAP, DSPE-PEG2000, and cholesterol.
^{b}The lipid in the shell contains DOPC, DOPA, DSPE-PEG2000, and cholesterol.
^{c}The lipid in the shell contains DOPC, DOPA, DSPE-PEG2000, cholesterol, and PLGA.

### Examples A18

### Preparation of CX-1 Nanoparticles Containing Tannic Acid

Nanoparticle A18 of this invention was prepared following the procedure as follows. A CX-1 aqueous solution (20 mg/mL, 50 μL) was mixed with 250 μL of a tannic acid aqueous solution (20 mg/mL) to form cores containing tannic acid and CX-1. After centrifuging at 25000 g for 15 minutes, the cores were collected and then dissolved in 400 μL of DMSO, to which was added 264 μL of a DMSO solution containing 1,2-dioleoyl-sn-glycero-3-phosphocholine (DOPC) (0.6 mg), DSPE-PEG2000 (1.29 mg), cholesterol (0.6 mg), and PLGA 50/50 (12 mg) to obtain 664 μL of an organic phase. Subsequently, the organic phase was added dropwise to 4.64 mL of water (volume ratio of oil and water, 1/7). Nanoparticles were formed after 20 cycles of sonication for 1 minute and 40 seconds on an ice bath. Each cycle included seconds of sonication pulse followed by a pulse-off period of 5 seconds, using a Q125 sonicator (Qsonica, Newtown, Connecticut). The resultant emulsion was centrifuged at 25,000 g and 25° C. for 20 minutes to afford a pharmaceutical nanoparticle of this invention, i.e., Nanoparticle A18, which was dispersed in 400 μL of water to obtain a Nanoparticle A18 aqueous dispersion ready for injection.

### Examples B1-B5

### Preparation of CX-1 Liposomes

Liposome B5 of this invention was prepared as follows. A thin film was prepared by evaporating the solvent in a solution containing 64 mg of DSPC, 15.68 mg of cholesterol, and 3.08 mg of DSPE-PEG2000 in 1 mL of chloroform. It was then hydrated using 1 mL of 300 mM ammonium sulfate buffer containing 9 mg of CX-1 diphosphate. The resultant mixture was vortexed at 25° C. for 5 minutes and then shaken at 65° C. for 60 minutes, followed by five freeze-thaw cycles using liquid nitrogen and 65° C. water bath alternatingly. Subsequently, it was extruded through a polycarbonate membrane (pore diameter: 100 nm) for 13 times at 65° C. using a mini-extruder device. The extruded mixture was centrifuged through a 100K Amicon® Ultra 0.5 mL Centrifugal Filter at 4° C. to afford a liposome of this invention, i.e., Liposome B5, which was dispersed in an HBS buffer (a pH value of 7.4) containing 10 mM HEPES and 140 mM NaCl.

Four more liposomes of this invention, i.e., B1-B4, were prepared following the above-described procedure except that different amounts of CX-1, DSPC, cholesterol, and DSPE-PEG2000 were used. See Tables 4 and 4a below.

TABLE 4
Liposomes B1-B5

| | CX-1 | DSPC | Cholesterol | DSPE-PEG2000 |
|---|---|---|---|---|
| Example | (mg) | (mg) | (mg) | (mg) |
| B1 | 36 | 32 | 7.84 | 1.54 |
| B2 | 18 | 32 | 7.84 | 1.54 |
| B3 | 9 | 16 | 3.92 | 0.77 |
| B4 | 9 | 32 | 7.84 | 1.54 |
| B5 | 9 | 64 | 15.7 | 3.08 |

TABLE 4a
Weight ratios

| | Weight ratio | | | |
|---|---|---|---|---|
| Example | CX-1 | DSPC | cholesterol | MPEG2000-DSPE |
| B1 | 1 | 0.89 | 0.22 | 0.04 |
| B2 | 1 | 1.78 | 0.44 | 0.09 |
| B3 | 1 | 1.78 | 0.44 | 0.09 |
| B4 | 1 | 3.56 | 0.87 | 0.17 |
| B5 | 1 | 7.11 | 1.74 | 0.34 |

### Entrapment Efficiency

Entrapment efficiencies were measured for Nanoparticles A1-A17 and Liposomes B1-B5.

The entrapment efficiency (EE %) was calculated using following equations:

$$EE ( \% ) = \frac{Total amount of drug - amount of unencapsulated drug}{Total amount of drug} \times 100 \% .$$

Three methods were followed to obtain EE %.

A first method was used to calculate EE % for each of Nanoparticles A1-A8. A supernatant was collected after centrifugation but before dissolving CX-1 cores into chloroform. CX-1 in the supernatant was unencapsulated. Its concentration was measured by high-performance liquid chromatography (HPLC). The CX-1 cores and the lipid obtained from the centrifugation were dried and then resuspended in water. The resultant mixture was centrifuged at 10000 g for 20 minutes. The supernatant was then collected and filtered using a centrifugal filter unit (100K Amicon® Ultra 0.5 mL, Millipore, Burlington, Massachusetts) at 14000 g for 10 min. The filtrate contained unencapsulated CX-1, the concentration of which was measured by HPLC. The unencapsulated CX-1 concentration was calculated from the HPLC results for each supernatant. The entrapment efficiency was obtained by calculating (the total amount of CX-1−the amount of unencapsulated CX-1) divided by the total amount of CX-1.

For Nanoparticles A9-A17, each nanoparticle sample was dissolved in a 10⁻² M NaOH aqueous solution to release all CX-1. Due to the overlap of absorbance wavelength below 270 nm of CX-1 and tannic acid, the concertation of tannic acid in the dissolved mixture was measured and calculated at 320 nm by a spectrometer (Multiskan® GO, ThermoFisher Scientific, Waltham, Massachusetts). The absorbance value of tannic acid at 270 nm was recalculated by the standard concentration of tannic acid with interpolation method. On the other hand, the absorbance value of CX-1 in the dissolved mixture at 270 nm was calculated by the total absorbance value of the dissolved mixture minus the absorbance value of tannic acid at 270 nm, thereby obtaining the amount of CX-1 in each nanoparticle sample. The entrapment efficiency was calculated as the amount of CX-1 in the nanoparticle divided by the total amount of CX-1 added to prepare the nanoparticle.

For Liposomes B1-B5, each of them was separated from unencapsulated CX-1 by centrifugation through a centrifugal filter unit (100K Amicon® Ultra 0.5 mL, Millipore, Burlington, Massachusetts). The filtrate contained unencapsulated CX-1. The liposome having the encapsulated CX-1 was collected and disintegrated with ethanol to a final concentration of 70% v/v. The CX-1 concentrations in both the filtrate and the liposome were determined by HPLC at 254 nm. The entrapment efficiency was calculated as the amount of CX-1 in the liposome divided by the total amount of CX-1 in the liposome and the filtrate.

The results show EE % in the range of 8% to 65.6%, see e.g., Table 5 below.

TABLE 5
Entrapment efficiencies (EE)

| Example | EE, % |
|---|---|
| A2 | 65.6 |
| A5 | 51.2 |
| A6 | 57.2 |
| A7 | 60.7 |
| A16 | 49.6 |
| A17 | 91.2 |
| B1 | 12.8 |
| B2 | 19.2 |
| B3 | 8.1 |
| B4 | 18.7 |
| B5 | 24.9 |

### Particle Size and Polydispersity Index

Particle sizes and polydispersity indexes (“PDIs”) were measured for Nanoparticles A1-A18 and Liposomes B1-B5.

The particle size and PDI of each of Nanoparticles A1-A18 were measured as follows. Each nanoparticle was formulated as described above and resuspended in water with 4-fold dilution. It was then sonicated for a total of 1 minutes and 40 seconds in an ice bath. Each cycle included 5 seconds of sonication pulse followed by a pulse-off period of 5 seconds (power, 40 W) with a Q125 sonicator (Qsonica, Newtown, Connecticut). The thus-obtained nanoparticle sample was added to a spectrophotometer cuvette for measurement. The particle size and PDI were obtained using a Zetasizer® system (Zetasizer® nano zs, Malvern Instruments Ltd., Worcestershire, UK) at room temperature.

The particle size and PDI of each of Liposomes B1-B5 were measured by dynamic light scattering (Zetasizer® Nano-ZS, Malvern, UK). A helium-neon (He—Ne) ion laser at 633 nm was used as the incident beam. The detection angle and temperature were 1730 and 25° C., respectively. Each sample was placed in a specimen holder 40 seconds prior to measurement to allow equilibration to room temperature.

The results are shown in Tables 6 and 7 below.

TABLE 6

| | Particle | | | | Loaded |
|---|---|---|---|---|---|
| | size | | Zeta | | CX-1 |
| Example | (nm) | PDI^{a} | potential(mV) | L.C.(%)^{b} | (μg) |
| A1 | 253.4 | 0.317 | −16.7 | 9.5 | 228.3 |
| A2 | 274.6 | 0.35 | −20.2 | 3.1 | 65.6 |
| A3 | 113.7 | 0.321 | −10.4 | 5.2 | 117.5 |
| A4 | 117.1 | 0.353 | −15.6 | 14.1 | 389.6 |
| A5 | 123.8 | 0.373 | −15.7 | 16.6 | 511.5 |
| A6 | 168.5 | 0.269 | −19.1 | 17.2 | 572 |
| A7 | 124 | 0.256 | −22 | 17 | 607.3 |
| A8 | 150.6 | 0.315 | −22.3 | 21.6 | 765 |

^{a}PDI: polydispersity index;
^{b}Loading capacity

TABLE 7

| | Particle | | Zeta |
|---|---|---|---|
| Example | size (nm) | PDI | potential(mV) |
| A9 | 153.9 | 0.168 | −9.44 |
| A10 | 174.6 | 0.188 | −12.93 |
| A11 | 179.9 | 0.166 | −10.9 |
| A12 | 192.3 | 0.18 | −15.5 |
| A13 | 151.6 | 0.205 | −18.63 |
| A14 | 176.5 | 0.176 | −17.83 |
| A15 | 191.8 | 0.196 | −20.03 |
| A16 | 290.7 | 0.352 | −20.0 |
| A17 | 188.2 | 0.3775 | −38.05 |
| A18 | 171.8 | 0.124 | −23.0 |
| B1 | 143.8 | 0.012 | −6.76 |
| B2 | 150.7 | 0.035 | −4.07 |
| B3 | 156.2 | 0.065 | −3.97 |
| B4 | 144.8 | 0.051 | −6.13 |
| B5 | 156.4 | 0.052 | −4.14 |

### Zeta Potential

Zeta potentials were measured as follows.

For Nanoparticles A1-A18, each nanoparticle was formulated as described above, resuspended in water to 4-fold dilution, sonicated for a total of 1 minutes and 40 seconds, and added to a folded capillary zeta cell for measurement. Zeta potentials were examined using a Zetasizer® system (Zetasizer® nano zs, Malvern Instruments Ltd., Worcestershire, UK) at room temperature.

For Liposomes B1-B5, Zeta potentials were measured by dynamic light scattering (Zetasizer® Nano-ZS; Malvern, UK), using a helium-neon (He—Ne) ion laser at 633 nm and a detection temperature of 25° C. Each sample was placed in a specimen holder 40 seconds prior to measurement to allow equilibration to room temperature. Measured electrophoretic mobilities were converted to Zeta potentials using the Smoluchowski's formula. The results are shown in Tables 6 and 7 above.

### Loading Capacity

Loading capacities (LC %) were calculated as the amount of total entrapped CX-1 (i.e., the amount of total CX-1 times the entrapment efficiency) divided by the theoretical total nanoparticle weight (i.e., the amount of total CX-1 times the entrapment efficiency+the amount of calf DNA+the amount of inner and outer lipids).

The results are shown in Table 6 above.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A pharmaceutical nanoparticle comprising a core and a shell coating the core, wherein the core contains (3-{4-[2-({4-[3-(3-cyclohexylaminopropyl-amino)propyl]-oxazol-2-ylmethyl}-amino)-6-methyl-pyrimidin-4-ylamino]-piperidin-1-yl}-3-oxo-propylamino)-acetic acid or a salt thereof (“CX-1”), 1,2-dioleoyl-sn-glycero-3-phosphate (“DOPA”), and an anionic polymer; and the shell contains a lipid.

2. The pharmaceutical nanoparticle of claim 1, wherein CX-1 is (3-{4-[2-({4-[3-(3-cyclohexylaminopropylamino)propyl]-oxazol-2-ylmethyl}-amino)-6-methyl-pyrimidin-4-ylamino]-piperidin-1-yl}-3-oxo-propylamino)-acetic acid diphosphate.

3. The pharmaceutical nanoparticle of claim 1, wherein the weight ratio of CX-1:the lipid is 1:80 to 4:1, preferably 1:40 to 2:1, and more preferably 1:20 to 1:1.

4. The pharmaceutical nanoparticle of claim 1, wherein the nanoparticle has a particle size of 1 nm to 1000 nm, preferably 10 nm to 500 nm, and more preferably 100 nm to 300 nm.

5. The pharmaceutical nanoparticle of claim 1, wherein the nanoparticle has a zeta potential of 0 mV to −100 mV, preferably −1 mV to −50 mV, and more preferably −5 mV to −30 mV.

6. The pharmaceutical nanoparticle of claim 1, wherein the core further contains 1,2-dioleoyl-3-trimethylammonium-propane (“DOTAP”).

7. The pharmaceutical nanoparticle of claim 1, wherein the anionic polymer is calf thymus deoxyribonucleic acid (“DNA”), a polyphenol, cyclic guanosine monophosphate-adenosine monophosphate (“cGAMP”), a small interfering ribonucleic acid (“siRNA”), or a plasmid DNA.

8. The pharmaceutical nanoparticle of claim 7, wherein the anionic polymer is calf thymus DNA, and the weight ratio of CX-1:DOPA:calf thymus DNA is 1:(0.01-100):(0.01-100), preferably 1:(0.05-20):(0.05-20), and more preferably 1:(1-20):(0.4-1).

9. The pharmaceutical nanoparticle of claim 1, wherein the anionic polymer is a polyphenol.

10. The pharmaceutical nanoparticle of claim 9, wherein the polyphenol is selected from the group consisting of tannic acid, 1,2,3,4,6-pentagalloyl glucose, epigallocatechin gallate, 0-glucogallin, 3,4,5-trihydroxybenzoic acid, Theaflavin-3-gallatt, raspberry ellagitannin, acertannin, and hamamelitannin.

11. The pharmaceutical nanoparticle of claim 10, wherein the anionic polymer is tannic acid, and the weight ratio of CX-1:DOPA:tannic acid is 1:(0.01-100):(0.01-100), preferably 1:(0.05-20):(0.05-20), and more preferably 1:(1-4):(1-10).

12. The pharmaceutical nanoparticle of claim 1, wherein the lipid is cholesterol, 1,2-dioleoyl-sn-glycero-3-phosphate (“DOPA”), 1,2-dioleoyl-sn-glycero-3-phosphocholine (“DOPC”), 1,2-dioleoyl-sn-glycero-3-phosphoethanol-amine (“DOPE”), 1,2-dioleoyl-3-trimethylammonium-propane (“DOTAP”), 1,2-distearoyl-sn-glycero-3-phosphoethanolamine-N-[methoxy(polyethylene glycol)] (“DSPE-PEG”), poly(D,L-lactide-co-glycolide) (“PLGA”), or any combination thereof.

13. The pharmaceutical nanoparticle of claim 12, wherein the lipid is selected from one or more of 1,2-distearoyl-sn-glycero-3-phosphoethanolamine-N-[methoxy(polyethylene glycol)-2000] (“DSPE-PEG2000”), DOPC, DOTAP, cholesterol, and PLGA; and the weight ratio of DSPE-PEG2000:DOPC:DOTAP:cholesterol:PLGA is 4:(0-10):(0-10):(0-10):(0-10), preferably 4:(0.2-5):(0.2-5):(0.2-5):(0-5), and more preferably 4:(0.5-2):(0.5-2):(0.5-2):(0-0.2).

14. The pharmaceutical nanoparticle of claim 12, wherein the lipid is selected from one or more of 1,2-distearoyl-sn-glycero-3-phosphoethanolamine-N-[methoxy(polyethylene glycol)-2000] (“DSPE-PEG2000”), DOPC, DOPA, cholesterol, and PLGA; and the weight ratio of DSPE-PEG2000:DOPC:DOPA:cholesterol:PLGA is 4:(0-10):(0-10):(0-10):(0-10), preferably 4:(0.2-5):(0.2-5):(0.2-5):(0-5), and more preferably 4:(0.5-2):(0.5-2):(0.5-2):(0-0.2).

15. A method of preparing a pharmaceutical nanoparticle of claim 1, the method comprising the steps of: providing a core dispersion having cores dispersed in a solvent, the cores each containing CX-1, DOPA, and an anionic polymer; providing a lipid, mixing the core dispersion and the lipid, thereby coating each of the cores with the lipid.

16. A liposome comprising a lipid bilayer enclosing an aqueous core, wherein the lipid bilayer contains 1,2-distearoyl-sn-glycero-3-phosphocholine (“DSPC”), cholesterol, and DSPE-PEG2000; and the aqueous core contains (3-{4-[2-({4-[3-(3-cyclohexylamino-propylamino)-propyl]-oxazol-2-ylmethyl}-amino)-6-methyl-pyrimidin-4-ylamino]-piperidin-1-yl}-3-oxo-propylamino)-acetic acid or a salt thereof (“CX-1”).

17. The liposome of claim 16, wherein the liposome has a particle size in diameter of 30 nm to 300 nm, preferably 100 nm to 200 nm, and more preferably 140 nm to 160 nm.

18. The liposome of claim 16, wherein the liposome has a zeta potential of 0 mV to −20 mV, preferably, −1 mV to −15 mV, and more preferably −2 mV to −10 mV.

19. The liposome of claim 16, wherein the weight ratio of CX-1:DSPC:cholecterol:DSPE-PEG2000 is 1:(0.5-12):(0.1-4):(0.02-1), preferably 1:(4-10):(0.5-2.5):(0.1-0.5), and more preferably 1:(6-8):(1.5-2):(0.3-0.4).

20. A method of preparing a liposome of claim 16, the method comprising the steps of: providing a thin film containing DSPC, cholesterol, and DSPE-PEG2000, mixing the thin film with an aqueous solution containing CX-1 and ammonium sulfate to obtain a hydration mixture, freezing the hydration mixture to a temperature of −150° C. to −200° C. and then thawing it to a temperature of 50° C. to 75° C. to obtain a dispersion containing the liposome.

21. The method of claim 20, further comprising the step of extruding the dispersion through a membrane having a pore diameter of 30 to 400 nm.

22. The method of claim 21, wherein the membrane is formed of polycarbonate.

23. The method of claim 20, wherein the freezing-thawing step is repeated 4 to 10 times.

24. The method of claim 20, wherein the aqueous solution contains 0.3 wt % to 8 wt % of CX-1 and 1 wt % to 6 wt % of ammonium sulfate.
